(19) United States
(12) Patent Application Publication
(10) Pub. No.: US 2025/0064756 A1
(43) Pub. Date: Feb. 27, 2025

(54) NANOCRYSTALLINE TIO2 AND ZNO ORGANOSELENIUM SCAFFOLDS POSSESING DISELENIDE (SE-SE) FUNCTIONALITY FOR BIOMEDICAL TECHNOLOGY

(71) Applicant: KING FAISAL UNIVERSITY, Al-Ahsa (SA)

(72) Inventors: Hany Mohamed Abd El-Lateef AHMED, Al-Ahsa (SA); Saadeldin Elsayed Ibrahim SHABAAN, Al-Ahsa (SA); Mai Mustafa Khalaf ALI, Al-Ahsa (SA); Mohamed GOUDA, Al-Ahsa (SA); Ibrahim Mohamed Abdelhalim MOHAMED, Sohag (EG)

(21) Appl. No.: 18/237,978

(22) Filed: Aug. 25, 2023

(51) Int. Cl. A61K 31/136; A61K 9/16; A61K 31/196; A61K 33/24; A61K 33/30; A61P 31/04

(52) U.S. Cl. CPC A61K 31/136; A61K 9/16; A61K 31/196; A61K 33/24; A61K 33/30; A61P 31/04; B82Y 5/00; B82Y 40/00

(57) ABSTRACT

Novel composites as antibacterial agents having crystalline oxides (TiO₂ and ZnO) combined with organoselenium compounds. The organoselenium aids in increasing the biosafety of the synthesized composite, which is characterized by different physicochemical techniques. Additionally, XRD indicates that the oxide part of the introduced composite is a mixture of TiO₂ as a rutile structure and ZnO as a hexagonal structure. The results confirm the successful grafting of the organoselenium compound onto the mixed oxide nanoparticles. Besides, the SEM and TEM morphology of the fabricated composites was found as heterogeneous particles that are formed as micro flowers or nanosheets according to the specific organoselenium compound used. The formed composites have a promising inhibition zone against many microorganisms and can be effective as novel antibacterial agents.

### BACKGROUND

### 1. Field

The disclosure of the present patent application relates to TiO₂/ZnO (ZT) organoselenium scaffolds possessing diselenide (Se—Se) functionality.

### 2. Description of the Related Art

Current antifungal, antibacterial, antioxidant, antiaging, anti-wrinkle, wound healing and similar therapies suffer from a number of limitations. For example, while adsorption of various treatment agents onto nanocarriers, such as ZnO or TiO₂, are known, such treatments have many limitations, including limited exposure time, high application frequency, high relapse rates, and limited efficacy.

Therefore, developing new TiO₂ and ZnO products solving the aforementioned problems are highly desired.

### SUMMARY

The present subject matter relates to bioxides of TiO₂ and ZnO combined with organic scaffolds as promising substrates for different applications, including in the biomedical field. Accordingly, the present subject matter relates to novel materials based on TiO₂/ZnO (ZT)@organoselenium scaffolds possessing diselenide (Se—Se) functionality.

Two diaryl diselenides (A and B) are synthesized and combined with a ZT composite to form A(ZT) and B(ZT) composites and their antimicrobial activities are evaluated against a variety of microorganisms. The prepared ZT-composites are chemically studied by different physicochemical techniques to define their surface chemical contents and morphological properties as well as the functional group analysis via FT-IR and thermal analysis. The morphology of the fabricated A(ZT) and B(ZT) as oxide/organoselenium composites have heterogeneous particles that seem to be micro flowers in the case of using compound A and nanosheets in the case of compound B. These composites provide a promising strategy for presenting low-cost antimicrobial agents for biomedical technology.

In another embodiment, the present subject matter relates to a bi-oxide substrate having TiO₂ and ZnO introduced at organoselenium compounds for presenting novel antimicrobial agents. The introduced organoselenium oxide composites are investigated on morphological character via TEM and FESEM in addition to surface chemistry analysis using the XPS technique to deeply understand their chemistry and morphology. After that, the antimicrobial performance against a variety of microorganisms is evaluated and it is found that the oxide/organoselenium composite is a promising antibacterial agent.

In an embodiment, the present subject matter relates to a nanocrystalline composite, the nanocrystalline composite comprising a bioxide of TiO₂ and ZnO (ZT) nanoparticles combined with a diaryl diselenide to form a TiO₂/ZnO (ZT)@organoselenium composite possessing diselenide functionality. In this regard, the diaryl diselenide in the nanocomposite can be selected from the group consisting of:

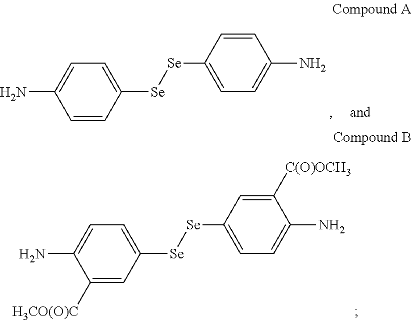

and the nanocrystalline composite is A(ZT) or B(ZT).

In another embodiment, the present subject matter relates to a method of treating a microbial infection in a patient, the method comprising administering to a patient in need thereof a therapeutically effective amount of the nanocrystalline composite as described herein.

In a further embodiment, the present subject matter relates to a method of making the nanocrystalline composite as described herein, the method comprising: sonicating the diaryl diselenide in an aqueous ethanolic solvent; stirring the sonicated diaryl diselenide in the aqueous ethanolic solvent to obtain a solution; dispersing TiO₂ and ZnO in the solution to obtain a dispersion; stirring the dispersion at room temperature overnight to obtain a suspension; and filtering the suspension to isolate and obtain the nanocrystalline composite.

These and other features of the present subject matter will become readily apparent upon further review of the following specification.

### BRIEF DESCRIPTION OF DRAWINGS

FIGS. 1A and 1B are SEM images of the synthesized A(ZT) (FIG. 1A) and B(ZT) (FIG. 1B) composites.

FIGS. 2A and 2B are TEM images of the synthesized A(ZT) (FIG. 1A) and B(ZT) (FIG. 1B) composites.

Similar reference characters denote corresponding features consistently throughout the attached drawings.

### DETAILED DESCRIPTION OF THE PREFERRED EMBODIMENTS

The following definitions are provided for the purpose of understanding the present subject matter and for construing the appended patent claims.

### Definitions

Throughout the application, where compositions are described as having, including, or comprising specific components, or where processes are described as having, including, or comprising specific process steps, it is contemplated that compositions of the present teachings can also consist essentially of, or consist of, the recited components, and that the processes of the present teachings can also consist essentially of, or consist of, the recited process steps.

It is noted that, as used in this specification and the appended claims, the singular forms “a”, “an”, and “the” include plural references unless the context clearly dictates otherwise.

In the application, where an element or component is said to be included in and/or selected from a list of recited elements or components, it should be understood that the element or component can be any one of the recited elements or components, or the element or component can be selected from a group consisting of two or more of the recited elements or components. Further, it should be understood that elements and/or features of a composition or a method described herein can be combined in a variety of ways without departing from the spirit and scope of the present teachings, whether explicit or implicit herein.

The use of the terms “include,” “includes”, “including,” “have,” “has,” or “having” should be generally understood as open-ended and non-limiting unless specifically stated otherwise.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ±10% variation from the nominal value unless otherwise indicated or inferred.

The term “optional” or “optionally” means that the subsequently described event or circumstance may or may not occur, and that the description includes instances where said event or circumstance occurs and instances in which it does not.

Unless defined otherwise, all technical and scientific terms used herein have the same meaning as commonly understood to one of ordinary skill in the art to which the presently described subject matter pertains.

Where a range of values is provided, for example, concentration ranges, percentage ranges, or ratio ranges, it is understood that each intervening value, to the tenth of the unit of the lower limit, unless the context clearly dictates otherwise, between the upper and lower limit of that range and any other stated or intervening value in that stated range, is encompassed within the described subject matter. The upper and lower limits of these smaller ranges may independently be included in the smaller ranges, and such embodiments are also encompassed within the described subject matter, subject to any specifically excluded limit in the stated range. Where the stated range includes one or both of the limits, ranges excluding either or both of those included limits are also included in the described subject matter.

Throughout the application, descriptions of various embodiments use “comprising” language. However, it will be understood by one of skill in the art, that in some specific instances, an embodiment can alternatively be described using the language “consisting essentially of” or “consisting of”.

“Subject” as used herein refers to any animal classified as a mammal, including humans, domestic and farm animals, and zoo, sports, and pet companion animals such as household pets and other domesticated animals such as, but not limited to, cattle, sheep, ferrets, swine, horses, poultry, rabbits, goats, dogs, cats and the like.

“Patient” as used herein refers to a subject in need of treatment of a condition, disorder, or disease, such as a microbial infection.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about”. Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

The present subject matter relates to bioxides of TiO₂ and ZnO combined with organic scaffolds as promising substrates for different applications, including in the biomedical field. Accordingly, the present subject matter relates to novel materials based on TiO₂/ZnO (ZT)@organoselenium scaffolds possessing diselenide (Se—Se) functionality.

Two diaryl diselenides (A and B) can be synthesized and combined with a ZT composite to form A(ZT) and B(ZT) composites and their antimicrobial activities can be evaluated against a variety of microorganisms. The prepared ZT-composites can be chemically studied by different physicochemical techniques to define their surface chemical contents and morphological properties as well as the functional group analysis via FT-IR and thermal analysis. The morphology of the fabricated A(ZT) and B(ZT) as oxide/organoselenium composites can have heterogeneous particles that seem to be micro flowers in the case of using compound A and nanosheets in the case of compound B. These composites can provide a promising strategy for presenting low-cost antimicrobial agents for biomedical technology.

In another embodiment, the present subject matter relates to a bi-oxide substrate having TiO₂ and ZnO introduced at organoselenium compounds for preparing novel antimicrobial agents. The introduced organoselenium oxide composites can be investigated on morphological character via TEM and SEM in addition to surface chemistry analysis using the XPS technique to deeply understand their chemistry and morphology. After that, the antimicrobial performance against a variety of microorganisms can be evaluated to find that the oxide/organoselenium composite is a promising antibacterial agent.

In an embodiment, the present subject matter relates to a nanocrystalline composite, the nanocrystalline composite comprising a bioxide of TiO₂ and ZnO (ZT) nanoparticles combined with a diaryl diselenide to form a TiO₂/ZnO (ZT)@organoselenium composite possessing diselenide functionality. In this regard, the diaryl diselenide in the nanocomposite can be selected from the group consisting of:

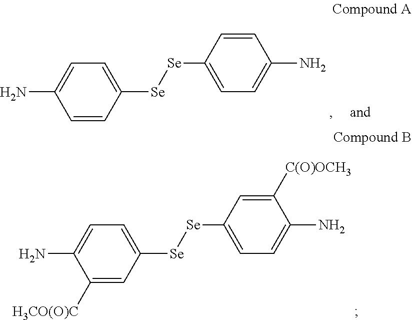

and the nanocrystalline composite is A(ZT) or B(ZT).

In one embodiment, the nanocrystalline composite can be A(ZT) and can comprise nanosized heterogenous particles which agglomerate to form micro flowers. In another embodiment, the nanocrystalline composite can be B(ZT) and can comprise heterogenous particles formed as nano-thin sheets.

In a further embodiment, the ZT nanoparticles can accumulate at a surface of compound B.

In an additional embodiment, the nanocrystalline composites A(ZT) and B(ZT) can be more porous than the ZT nanoparticles.

Metal-based diselenides are usually used as channel materials to overcome challenges facing silicon-based instruments owing to the metal and selenium compact lattice structures. Diaryl diselenides A and B can be synthesized in two steps starting with the selenocyanation of aniline (A) or methyl 2-aminobenzoate (B) using triselenium dicyanide to give the corresponding 4-selenocyanatoaniline (A) and methyl 2-amino-5-selenocyanatobenzoate (B), respectively. Hydrolysis of the selenocyanates A and B using NaOH can provide the corresponding diaryl diselenides A and B in 82% and 92% yields, respectively.

In an embodiment, the TiO₂ can be present in the nanocrystalline composite as a rutile structure and the ZnO can be present in the nanocrystalline composite as a hexagonal structure.

In another embodiment, the present subject matter relates to a method of treating a microbial infection in a patient, the method comprising administering to a patient in need thereof a therapeutically effective amount of the nanocrystalline composite as described herein.

In an embodiment, the diaryl diselenide used in the present methods of treatment can be selected from the group consisting of:

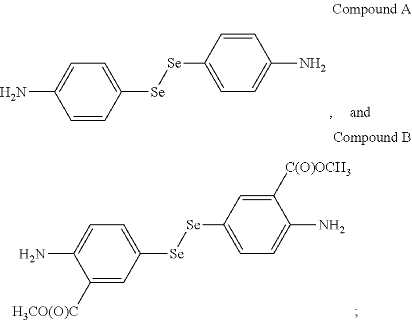

and the nanocrystalline composite can be A(ZT) or B(ZT).

In a further embodiment, the microbial infection can be caused by one or more of E. coli, S. aureus, C. Albicans, P. aeruginosa, A. flavus, and B. subtilis. In this regard, in instances where the nanocrystalline composite is A(ZT), the microbial infection can be caused by one or more of E. coli, S. aureus, and C. Albicans. Similarly, in instances where the nanocrystalline composite is B(ZT), the microbial infection can be caused by one or more of E. coli, S. aureus, P. aeruginosa, and B. subtilis.

In an additional embodiment, the present subject matter relates to a method of making the nanocrystalline composite as described herein, the method comprising: sonicating the diaryl diselenide in an aqueous ethanolic solvent; stirring the sonicated diaryl diselenide in the aqueous ethanolic solvent to obtain a solution; dispersing TiO₂ and ZnO in the solution to obtain a dispersion; stirring the dispersion at room temperature overnight to obtain a suspension; and filtering the suspension to isolate and obtain the nanocrystalline composite.

In certain embodiments, the sonicated diaryl diselenide in the aqueous ethanolic solvent can be stirred at a temperature of about 55° C. to about 65° C. for at least about 3 hours.

In other embodiments, the TiO₂ and the ZnO can be dispersed in the solution for at least about 3 hours.

The present teachings are illustrated by the following examples.

### EXAMPLES

All reagents and solvents used in these studies were purchased from Sigma and used without purification. Melting points (MP) were measured on the Gallenkamp apparatus in degrees centigrade. The IR spectra (KBr, λₘₐₓ·cm⁻¹) were recorded on a Mattson spectrophotometer (5000 FTIR) at King Faisal University. The ¹H & ¹³C NMR spectra were measured using Varian Spectrophotometer (400 MHz), employing DMSO-d6 as the solvent and TMS internal standard at Mansoura University. The chemical shifts (δ, ppm) were recorded regarding the solvent residual peaks. GC-MS-QP-100 EX Shimadzu apparatus was used for mass measurements at Cairo University. All biological tests were carried out at the Faculty of Pharmacy, Mansoura University. All cell lines and microorganisms were purchased from the VACSERA Company (ATCC), Cairo, Egypt. DPPH and ABTS probes were obtained from Sigma.

### Example 1

### Synthesis of Diaryl Diselenide A

Diaryl diselenide A was synthesized from aniline (1) as shown in Scheme 1:

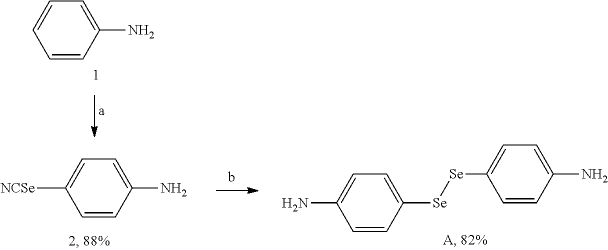

Compound A was prepared by combining benzenamine (1) (2.4 mmol), propanedinitrile (1.6 mmol), SeO₂ (3.2 mmol) and DMSO (2 mL) to obtain 4-selenocyanatoaniline (2); and then combining 4-selenocyanatoaniline (2) (1 mmol), NaOH (1.5 mmol), and ethanol (20 mL) to obtain compound A in a yield of about 82%.

### Example 2

### Synthesis of Diaryl Diselenide B

Diaryl diselenide B was synthesized from methyl 2-aminobenzoate (4) as shown in Scheme 2:

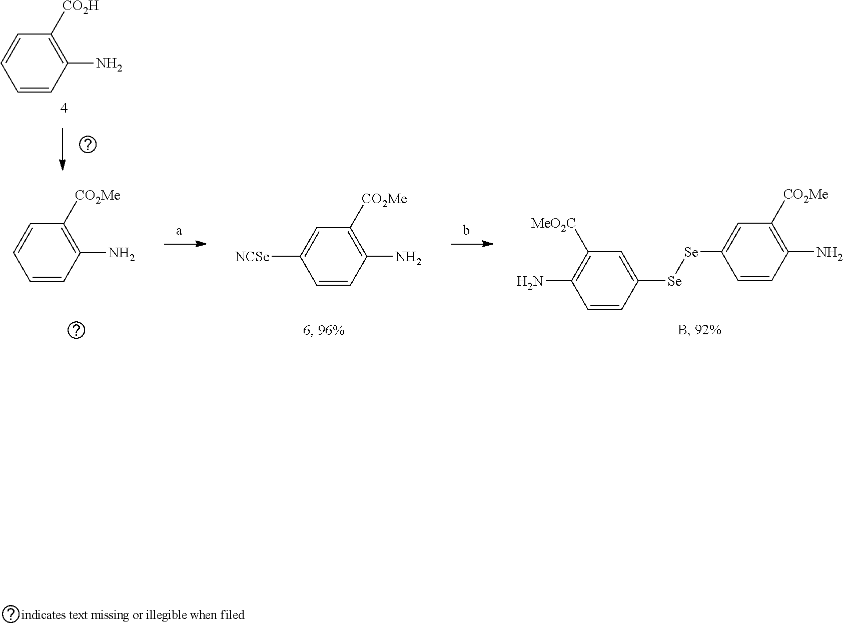

Compound B was prepared by combining 2-aminobenzoic acid (4) (15.55 mmol), methanol (30 mL), and concentrated H₂SO₄ (2 mL) with reflux for 42 h; to obtain methyl 2-aminobenzoate (5). Next, propanedinitrile (1.6 mmol), SeO₂ (3.2 mmol), DMSO (10mL), and methyl 2-aminobenzoate (5) (2.4 mmol) were combined to obtain methyl 2-amino-5-selenocyanatobenzoate (6); and then methyl 2-amino-5-selenocyanatobenzoate (6) (1 mmol), NaOH (1 mmol), and ethanol (20 mL) were combined to obtain compound B in a yield of about 92%.

More specifically, methyl 2-amino-5-selenocyanatobenzoate (6) was synthesized from the reaction of methyl 2-aminobenzoate with triselenium dicyanide prepared in situ from malononitrile and selenium dioxide in 96% yields. Briefly, selenium dioxide (30 mmol, 3300 mg) was added to malononitrile (15 mmol, 1000 mg) in 10 mL DMSO, and the mixture was stirred for 20 minutes at room temperature. Next, the mixture was filtered off to get rid of any formed black selenium, and methyl 2-aminobenzoate (12.5 mmol, 1800 mg) was then added, and the reaction mixture was stirred for a further 2 hr at room temperature. Finally, adding 10 g of ice terminated the reaction, and the formed precipitate was filtered, washed several times with H₂O and sodium carbonate solution, dried, and recrystallized from petroleum ether. Additionally, compound dimethyl 5,5-diselanediylbis(2-aminobenzoate) (B) (7) was synthesized from the reaction of 6 and sodium hydroxide in 92% yields.

Briefly, compound 6 (4 mmol, 1000 mg) was dissolved in EtOH (20 mL), and then sodium hydroxide (4 mmol, 160 mg) was added. The reaction mixture was stirred for 2 hours at room temperature, and the resulting precipitate was filtered, washed several times with H₂O, and recrystallized from chloroform.

Methyl 2-amino-5-selenocyanatobnzoate (6) was isolated as a reddish solid; yield: 3072 mg (96%); MP=118-119° C.; Rf=0.4 (petroleum ether/ethyl acetate 4:2). IR(KBr): λₘₐₓ·cm⁻¹:3475, 3366, 2946, 2147, 1691. ¹HNMR (400 MHz, DMSO-d6) δ8.02 (s, 1H, Ar-H), 7.57 (d, J=8.8 Hz, 1H, Ar-H), 7.08 (d, 1H, Ar-H), 6.84 (s, 2H, NH₂), 3.82 (s, 3H, OCH₃). ¹³C NMR (101 MHz, DMSO-d6) δ166, 152, 140, 137, 118, 109, 105, 105, 51. MS (EI, 70 ev) m/z (%)=259.35 (M+3H, 2.39), 117 (29.02), 87 (26.6), 75 (2.70), 59 (100.0, base peak).

Dimethyl 5,5′-diselanediylbis(2-aminobenzoate) (7) was isolated as a yellow solid; yield: 1692.43 mg (92%); MP=138-139° C.; Rf=0.5 (petroleum ether/ethyl acetate 4:3). IR(KBr): λₘₐₓ·cm⁻¹:3455, 3344, 2931, 1684. ¹HNMR (400 MHz, DMSO) δ7.70 (s,2H, Ar-H), 7.44 (d, J=8.6 Hz, 2H, Ar-H), 7.00 (s, 4H,2NH₂), 6.77 (d, J=8.7 Hz, 2H, Ar-H), 3.74 (s, 6H,2OCH₃). ¹³CNMR (101 MHz, DMSO-d6) δ167, 151, 140, 138, 117, 113, 108, 51. MS (EI, 70 ev) m/z (%)=460.15 (M+H, 20.76), 459.15 (M, 5.20) or 230 (24.42), 119 (9.45), 91 (100.0, base peak), 65 (8.88).

### Example 3

### Preparation of Nanocrystalline Composites

Bimetal oxides (TiO₂&ZnO) were attached to organoselenium Compound A and Compound B to fabricate novel oxide/active organics named A(ZT) and B(ZT) for using compound A and compound B, respectively.

The loading of the organoselenium was carried out by the simple methodology as follows: 2 mmol of organoselenium was sonicated in aqueous ethanolic solvent then stirred at 60° C. for 3 h. TiO₂ and ZnO were dispersed in the obtained solution for 3 h and followed by stirring at room temperature overnight to finally obtain the TiO₂ and ZnO@organoselenium composite. The formed suspension was filtered to isolate the introduced TiO₂ and ZnO@organoselenium composite.

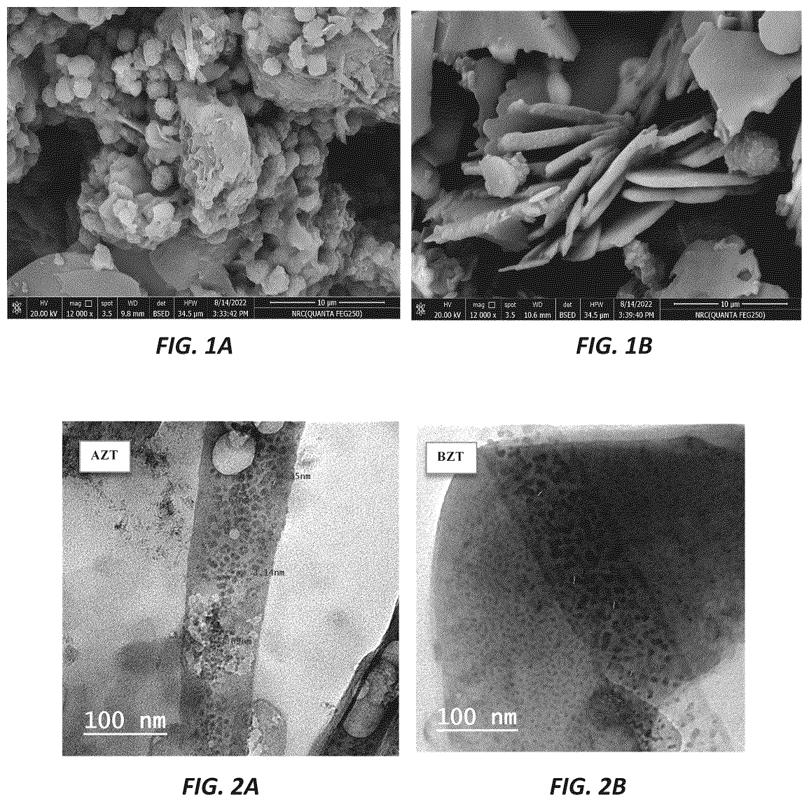

The difference between compound A and compound B is the carboxymethyl group and so, the impact of this group in morphological, crystallinity, and antimicrobial performance was investigated. The SEM morphology of the prepared ZT before interaction with organics was observed in FIGS. 1A and 1B for low and high magnifications, respectively. The observed SEM morphology has agglomerated particles having a nano-size. After that, the heterogeneity character of the designed A(ZT) particles becomes clearer and could be attributed to the organic part which provides a chance for particles to accumulate in a distinct shape.

The A(ZT) particles are nano-size and agglomerated to form micro flowers. Interestingly, the B(ZT) composite has nano-thin sheets which could be due to the accumulation of ZT nanoparticles at the surface of compound B which is larger than compound A. There are more pores in the case of A (ZT and B(ZT) if compared with ZT nanoparticles, which could be attributed to the heterogenicity of the morphology between particles and flowers in the case of A(ZT) and particles and sheets in the case of B(ZT).

The TEM images in FIGS. 2A and 2B indicate the formation of nanoparticles in the case of ZT without any organics in addition to the heterogeneous morphology in the case of both A(ZT) and B(ZT) composites. Some crystalline phases could be seen in all SAED images which could be attributed to the existence of oxides (TiO₂ and ZnO) inside the chemistry of the ZT, A(ZT), and B(ZT) composites. Besides, the ambiguous character of some seen ring circles could be detected because of organic compounds which traditionally have amorphous character. In short, the SEM and TEM morphology of the fabricated A(ZT) and B(ZT) as oxide/organoselenium composites have heterogeneous particles that seem to be micro flowers in the case of using compound A and nanosheets in the case of compound B.

### Example 4

### Antimicrobial Activity

In General, the diaryl diselenide B manifested promising antimicrobial activity against E. coli (A %=83%), S. aureus (A %=86%), and C. Albicans (A %=88%). On the other hand, fair activity was observed for diaryl diselenide A against S. aureus (A %=71%), E. coli (A %=54%), P. aeruginosa (A %=63 and 58), E. coli (A %=62 and 65), and B. subtilis (A %=65%). Unexpectedly, the A(ZT) and B(ZT) composites showed lower antimicrobial activity compared to the diaryl diselenides A and B. The results can be seen in Table 1.

| | E. coli | | P. aeruginosa | | S. aureus | | B. subtilis | | | | | |
|---|---|---|---|---|---|---|---|---|---|---|---|---|
| | (−ve) | | (−ve) | | (+ve) | | (+ve) | | C. albicans | | A. flavus | |
| Compounds | IZD^{a} | A % | IZD^{a} | A % | IZD^{a} | A % | IZD^{a} | A % | IZD^{a} | A % | IZD^{a} | A % |
| A | 14 | 54 | 11 | 48 | 17 | 71 | 15 | 65 | 12 | 44 | 8 | 32 |
| B | 19 | 83 | — | — | 18 | 86 | — | — | 21 | 88 | — | — |
| A(ZT) | 9 | 35 | 8 | 35 | 12 | 50 | 10 | 44 | 9 | 33 | 7 | 28 |
| B(ZT) | 5 | 19 | 6 | 26 | 8 | 33 | 7 | 30 | 5 | 19 | 4 | 16 |
| Cip. | 26 | 100 | 23 | 100 | 24 | 100 | 23 | 100 | NA | — | NA | — |
| Col. | NA | — | NA | — | NA | — | NA | — | 27 | 100 | 25 | 100 |

^{a}IZD is the agar diffusion assay diameters in mm.
^{b}means no growth inhibition was noticed.

### Table 1. Antimicrobial Activity of the Diaryl Diselenides A and B

It is to be understood that the composites and methods are not limited to the specific embodiments described above, but encompass any and all embodiments within the scope of the generic language of the following claims enabled by the embodiments described herein, or otherwise shown in the drawings or described above in terms sufficient to enable one of ordinary skill in the art to make and use the claimed subject matter.

1. A nanocrystalline composite, the nanocrystalline composite comprising
a bioxide of TiO₂ and ZnO (ZT) nanoparticles combined with a diaryl diselenide to form a TiO₂/ZnO (ZT)@organoselenium composite possessing diselenide functionality.

2. The nanocrystalline composite of claim 1, wherein the diaryl diselenide is: 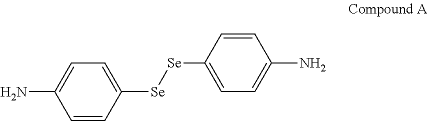 [SMILES: Nc1ccc([Se][Se]c2ccc(N)cc2)cc1]
; and
the nanocrystalline composite is A(ZT) or B(ZT).

3. The nanocrystalline composite of claim 2, wherein the nanocrystalline composite is A(ZT) and comprises nanosized heterogenous particles which agglomerate to form micro flowers.

4. The nanocrystalline composite of claim 2, wherein the nanocrystalline composite is B(ZT) and comprises heterogenous particles formed as nano-thin sheets.

5. The nanocrystalline composite of claim 4, wherein the ZT nanoparticles accumulate at a surface of compound B.

6. A method of treating a microbial infection in a patient, the method comprising administering to a patient in need thereof a therapeutically effective amount of the nanocrystalline composite of claim 1.

7. The method of treating a microbial infection of claim 8, wherein the diaryl diselenide is selected from the group consisting of: 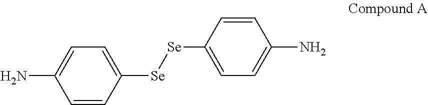 [SMILES: Nc1ccc([Se][Se]c2ccc(N)cc2)cc1]
; and
the nanocrystalline composite is A(ZT) or B(ZT).

8. The method of treating a microbial infection of claim 6, wherein the microbial infection is caused by one or more of E. coli, S. aureus, C. Albicans, P. aeruginosa, A. flavus, and B. subtilis.

9. The method of treating a microbial infection of claim 8, wherein the nanocrystalline composite is A(ZT) and the microbial infection is caused by one or more of E. coli, S. aureus, and C. Albicans.

10. The method of treating a microbial infection of claim 8, wherein the nanocrystalline composite is B(ZT) and the microbial infection is caused by one or more of E. coli, S. aureus, P. aeruginosa, and B. subtilis.

11. A method of making the nanocrystalline composite of claim 1, the method comprising:
sonicating the diaryl diselenide in an aqueous ethanolic solvent;
stirring the sonicated diaryl diselenide in the aqueous ethanolic solvent to obtain a solution;
dispersing TiO₂ and ZnO in the solution to obtain a dispersion;
stirring the dispersion at room temperature overnight to obtain a suspension; and
filtering the suspension to isolate and obtain the nanocrystalline composite.

12. The method of making the nanocrystalline composite of claim 11, wherein the sonicated diaryl diselenide in the aqueous ethanolic solvent is stirred at a temperature of about 55° C. to about 65° C. for at least about 3 hours.

13. The method of making the nanocrystalline composite of claim 11, wherein the TiO₂ and the ZnO are dispersed in the solution for at least about 3 hours.

14. The nanocrystalline composite of claim 2, wherein the nanocrystalline composites A(ZT) and B(ZT) are more porous than the ZT nanoparticles.

15. The nanocrystalline composite of claim 2, wherein the TiO₂ is present in the nanocrystalline composite as a rutile structure and the ZnO is present in the nanocrystalline composite as a hexagonal structure.
